(19) United States
(12) Patent Application Publication
(10) Pub. No.: US 2026/0364544 A1
(43) Pub. Date: Nov. 19, 2026

(54) METHOD FOR PREPARING A STABLE EMULSION BY MAGNETIC TURBULENCE

(71) Applicant: Ixchel Technologies, Bourges (FR)

(72) Inventors: Manon Desjardins, Henrichemont (FR); Marine Desjardins, Henrichemont (FR); Isabelle Desjardins-Lavisse, Henrichemont (FR)

(21) Appl. No.: 19/477,197

(22) PCT Filed: Apr. 16, 2024
(86) PCT No.: PCT/EP2024/060298

§ 371 Date: Oct. 21, 2025

(30) Foreign Application Priority Data
Apr. 21, 2023 (FR) FR2304020

(51) Int. Cl. B01F 23/411; A61K 8/06; B01F 23/41; B01F 101/21

(52) U.S. Cl. CPC B01F 23/411; B01F 23/4105; A61K 8/06; B01F 2101/21

(57) ABSTRACT

A method for preparing a stable emulsion from a mixture including at least one hydrophilic fraction and at least one hydrophobic fraction, at least one of the two fractions being liquid, the other being liquid or solid, by exclusively generating a turbulent flow applied to the mixture, the turbulent flow being generated and maintained by at least one magnetic force created by at least two identical magnetic poles.

### CROSS-REFERENCE TO RELATED APPLICATIONS

This application is a national phase entry under 35 U.S.C. § 371 of International Patent Application PCT/EP2024/060298, filed Apr. 16, 2024, designating the United States of America and published as International Patent Publication WO 2024/218090 A1 on Oct. 24, 2024, which claims the benefit under Article 8 of the Patent Cooperation Treaty of French Patent Application Serial No. FR2304020, filed Apr. 21, 2023.

### TECHNICAL FIELD

This present disclosure relates to a method for preparing a stable emulsion.

### BACKGROUND

The present disclosure relates more particularly to solutions or mixtures of the immiscible liquid type, colloidal solution (milk, low-fat cream, beverages derived from plants such as soy milk without the addition of stabilizing additives), active ingredients derived from plants in solid form or from a first extraction, in an aqueous, oily or alcoholic mixture, or a mixture thereof, solubilization of cosmetic active ingredients that are very poorly or not at all miscible in water.

Emulsion preparation processes are known. For example, an oil-in-water (O/W) or water-in-oil (W/O) emulsion is an unstable emulsion and tends to separate or coalesce.

Known methods use mechanical agitation to produce energy capable of dispersing the phase to be emulsified in the continuous medium and, depending on the target concentration, breaks it down into droplets, the size of which varies according to the type of emulsion: macro, nano or microemulsion.

The equipment used can be of the rotor-stator type, low-frequency ultrasound, high-pressure homogenizers or else micro-fluidizers.

In addition to this energy, stabilization is ensured by the use of one or more surfactants or substances that act together to perform this function. Thus, to ensure that the mixture of the two immiscible phases remains stable, an emulsifier is added. The basic principle for obtaining an O/W emulsion is therefore generally to finely disperse the lipid phase in water in the presence of an emulsifier.

Finally, it has been proposed in the publication by Stef Kerkhofs et al. “Mayonnaise production in batch and continuous process exploiting magnetohydrodynamic force” Journal of food engineering, Elsevier, Amsterdam, vol. 106, no. 1, Apr. 3, 2011, pages 35-39 to use a mechanical and magnetic system to achieve turbulence in a fluid, the system comprising two magnets: a magnet with north polarity and a magnet with south polarity. The system provides mechanical agitation in the presence of more than 10% emulsifying substances.

Nevertheless, it is keen to offer emulsions having improved properties, particularly in terms of stability and transparency, and/or that are simple to implement and energy-efficient.

### BRIEF SUMMARY

To this end, and according to a first aspect, the present disclosure proposes a method for preparing a stable emulsion from a mixture comprising at least one hydrophilic fraction and at least one hydrophobic fraction, at least one of the two fractions being liquid, the other being liquid or solid, by generating a turbulent flow applied to the mixture, the turbulent flow being generated and maintained by at least one magnetic force created by at least two identical magnetic poles.

The invention creates a special magnetic field that subjects a mixture to be emulsified in a pipe or container.

According to a first variant of the first aspect, the present disclosure proposes a method for preparing a stable emulsion from a mixture comprising at least one aqueous solution and at least one immiscible component naturally in the at least one aqueous solution, wherein the composition is placed in a preparation area and is subjected to at least one magnetic field produced by at least two identical magnetic poles facing each other.

According to a second variant of the first aspect, the present disclosure proposes a method for preparing an emulsion from at least one aqueous solution and at least one lipid element (solution or component) wherein the mixture of the aqueous solution and of the lipid element is placed between at least two magnetic poles facing one another, applying at least one magnetic field capable of creating turbulence in the mixture.

For the foregoing and for the rest of the present disclosure:
the at least one hydrophilic fraction can be liquid or solid;
the at least one hydrophobic fraction can be liquid or solid;
an immiscible component is defined as an element that is not soluble in water, or a solid, or an alcoholic solution, or an element or a lipid solution;
facing one another in combination with the magnetic poles, means an arrangement of the type facing each other symmetrically, or not, with respect to the geometric plane passing between the two poles;
turbulent flow means a turbulent flow of a liquid or of a mixture of liquids or of a mixture of at least one liquid with at least one solid, illustrated, for example, by the Reynolds number, the limit values of which between a laminar flow and a turbulent flow may vary according to the liquid(s) under consideration and that are known to the skilled person.

In fluid dynamics, turbulence is a flow regime characterized by chaotic changes in pressure and flow velocity. Turbulence is caused by excess kinetic energy in certain parts of the fluid flow, the excess kinetic energy counteracting the damping effect provided by the viscosity of the fluid.

A property of turbulent flow conventionally emphasized lies in a process known as turbulent cascade (or Richardson's cascade): the division of large vortices into smaller vortices enables a transfer of energy from large scales to small scales given by the Kolmogorov dimension. At the end of this transfer, the smallest vortices dissipate the energy they have received via a multitude of vortex cells whose number varies as the fourth root of the ninth power of the Reynolds number.

Herein, the innovative method is based on the application of incident energy to the water, to obtain a mesh of crystalline layers, capable of generating a new molecular order with a separation of charges capable of ensuring strong stability of the mixture, herein of emulsion of a lipid fraction in a continuous aqueous phase, see the following publication: Chai B, Pollack G 2010 “Solute-free interfacial zones in polar liquid.” The turbulent agitation he refers to in the innovative method is not of a physical nature but resembles a quantum phenomenon.

Quantum turbulence can be defined as a multi-scale and multi-physics phenomenon that appears in superfluid systems, such as superfluid helium and Bose-Einstein condensates (the superfluid transition can be seen as a Bose-Einstein condensation where a macroscopic fraction of helium atoms are in a single quantum state). This is a recent and dynamic field of research, a precursor of technological upheaval. It is based on the fact that, at finite temperature, two different fluids coexist and interact strongly: a viscous normal fluid described by the Navier Stokes equations and a viscosity-free superfluid described by the Gross-Pitaevskii equation. The presence of quantum vortices, generated in the superfluid fraction, is a feature that does not exist in conventional fluids.

Quantum turbulence differs from conventional turbulence in that the turbulent flow of a quantum fluid is confined around line-shaped flow centers called vortices, which can only take on quantized values.

These phenomena were demonstrated at very low temperatures by Bose and Einstein, but the work by Schouten, Sager-Smith and Maziotti of the University of Chicago published in 2023 shows that this phenomenon can also be observed at room temperature in plant photosynthesis, see publications (Exciton-Condensate-Like Amplification of Energy Transport in Light Harvesting, by Anna O. Schouten, LeeAnn M. Sager-Smith, and David A. Mazziotti published on Apr. 28, 2023 by American Physical Society and Julien Salort's thesis, “Quantum versus classical turbulence” publicly defended on Nov. 16, 2011).

According to other advantageous and non-limiting features of the present disclosure, taken alone or according to any technically feasible combination, in particular, with the first aspect and/or the first variant of the first aspect and/or the second variant of the first aspect:
the turbulent flow can be achieved without mechanical agitation, for example, without the device known as Ultra-Turrax®;
the turbulent flow can be obtained without mechanical agitation, without a fluid circulation system;
the turbulent regime can be generated and maintained solely by at least one magnetic force;
the turbulent flow can be obtained by a device arranged and configured to achieve quantum turbulence and/or the method corresponds to quantum turbulence;
the turbulent flow can be obtained exclusively by a magnetic force, preferably without a fluid circulation system that does not allow a Reynolds number (Re) proposed below to be obtained;
the turbulent flow can be obtained by dissipating at least one magnetic force, or Lorentz force, or Van der Waals force or at least one magnetohydrodynamic wave in the mixture;
the turbulent flow can be obtained by applying at least one magnetic field in the mixture so as to generate the turbulent flow, for example, with a Reynolds number (Re) greater than 1000, preferably greater than 2000 depending on the liquid(s);
preferably the turbulent flow is a high-intensity turbulent flow, for example, with a Reynolds number (Re) greater than 100,000, preferably greater than 200,000;
the turbulent flow can be achieved by dissipating the induction forces applied as kinetic energy, for example, with a Reynolds number (Re) greater than 100,000, preferably greater than 200,000;
the at least one magnetic field can be applied in the mixture so as to obtain a Reynolds number (Re) greater than 100,000, preferably greater than 200,000;
the turbulent flow can be generated and maintained by at least one magnetic force created by at least two magnetic poles positioned opposite each other;
the turbulent flow can be obtained by applying at least one magnetic field created by at least two magnetic poles positioned facing each other;
the at least two magnetic poles can be identical, either two north poles or two south poles;
the mixture can be placed equidistant from the at least two magnetic poles;
the fractions can be placed equidistant from the at least two magnetic poles;
the turbulent flow can be generated and maintained by at least one magnetic force or at least one magnetic field generating at least 1000 gauss, preferably at least 1500 gauss, preferably at least 1800 gauss, preferably at least 2000 gauss, preferably at least 2500 gauss, preferably at least 3000 gauss, preferably at least 3500 gauss, preferably at least 4000 gauss, preferably at least 4500 gauss, preferably at least 5000 gauss, preferably at least 5500 gauss, preferably at least 6000 gauss, preferably at least 6500 gauss, preferably at least 7000 gauss, preferably at least 8000 gauss, preferably at least 9000 gauss, preferably at least 10000 gauss, preferably at least 11000 gauss, preferably at least 12000 gauss, preferably at least 13000 gauss, preferably at least 14000 gauss, preferably at least 15000 gauss;
the turbulent flow can be generated and maintained by at least one magnetic force or at least one magnetic field generating at least 100 millitesla, preferably at least 150 millitesla, preferably at least 180 millitesla, preferably at least 200 millitesla, preferably at least 250 millitesla, preferably at least 300 millitesla, preferably at least 350 millitesla, preferably at least 400 millitesla, preferably at least 450 millitesla, preferably equal to 450 millitesla, preferably at least 500 millitesla, preferably at least 550 millitesla, preferably at least 600 millitesla, preferably at least 650 millitesla, preferably at least 700 millitesla, preferably at least 800 millitesla, preferably at least 900 millitesla, preferably at least 1000 millitesla;
preferably, the magnetic coercive force used can be greater than 200 KA/m, preferably greater than 300 KA/m, preferably greater than 400 KA/m, preferably greater than 500 KA/m, preferably greater than 600 KA/m, preferably greater than 700 KA/m, preferably greater than 800 KA/m, preferably greater than 850 KA/m, preferably greater than 900 KA/m, preferably greater than 1000 KA/m;
the at least two magnetic poles can be produced by permanent magnets, for example, comprising or consisting of neodymium or at least one electromagnet;
preferably the magnetic pole supports, or permanent magnets, have a U-shaped profile, for example, comprising or consisting of a ferromagnetic material, for example, steel, preferably mild steel;
according to one embodiment, a south pole of one magnet, referred to as the first magnet, can be located on the surface of the U-shaped support or opposite the other magnet, referred to as the second magnet, whereby the field lines of the north pole of the first magnet are deflected toward the surface by the flanks of the U-shaped profile. Similarly, if the north pole is at the surface of the U-shaped support, the field lines of the south pole will be directed toward the surface of the magnet; a support, also known as a cage, can thus be used to assemble permanent magnets in rows of identical north polarity, of identical south polarity or alternating north/south polarity;
preferably, according to one embodiment, two strictly identical cages are arranged in opposition so that each component is in opposition with its opposite component of the same polarity and of the same strength;
the mixture can be placed in a magnetically shielded enclosure, preferably in a plastic pipe;
preferably the container, for example, the pipe, extends between the at least two magnetic poles, preferably the at least two magnets or the at least two electromagnets;
preferably the container in the form of a pipe has a coil shape between the at least two magnetic poles;
the mixture can be in direct contact with the at least two magnetic poles;
the at least one magnetic field is applied until the size of the molecules or particles, in particular, the at least one hydrophobic fraction, is reduced by at least 20% by volume with respect to the initial size;
the at least one magnetic field is applied until the size of the molecules or particles is reduced to between 100 nanometers and 2 microns;
according to one variant, the mixture is static with respect to the at least two magnetic poles;
according to another variant, combinable with the previous variant, the mixture is movable with respect to the at least two magnetic poles, preferably using a pump to circulate the mixture in front of the at least two poles;
preferably no stabilizing agent is added to the mixture;
the product of the emulsification of immiscible phases can be obtained without the addition of a stabilizing agent;
preferably the mixture is additionally subjected to radiation, preferably of a light nature;
preferably the mixture is subjected to incident magnetic or electromagnetic energy coupled with light radiation;
preferably, the magnetic field exposure time can be proportional to the emulsion concentration;
according to one embodiment, the hydrophilic fraction is an aqueous solution and the hydrophobic fraction comprises at least one essential oil; and
the method is carried out without heating the mixture or without generating any heat.

Preferably, the method, according to one or more of the preceding characteristics, is intended for the preparation of a cosmetic or aromatic composition.

According to a second aspect, the present disclosure proposes a stable emulsion, obtained by implementing the method according to one or more of the preceding characteristics, wherein it is made up of particles greater than 400 nanometers in size.

The proposed solutions make it possible to obtain a particularly stable mixture and/or having one or more of the following properties: transparency, translucency, etc. These emulsions can be stable for up to several months, therefore making them suitable for industrial use.

According to one variant, the product of the emulsification of the immiscible phases can be an emulsion, known as a micro-emulsion, preferably a nano-emulsion, having a particle size of between 100 and 400 nm.

According to one variant, the product of the emulsification of the immiscible phases can be an emulsion, known as a micro-emulsion or nano-emulsion, having particle sizes of less than 100 nm without the addition of a stabilizer.

Preferably, grain or particle size can be measured using an optical or electron microscope or a granulometer.

According to one embodiment, the product of the emulsification of the immiscible phases can be an inverse emulsion.

Preferably, the method, according to one or more of the preceding features, is used for emulsifying at least one component in at least one aqueous solution.

### DETAILED DESCRIPTION

Conventionally, an emulsion is a homogeneous system comprising at least two immiscible liquids, one of which is dispersed in the other in a more or less stable form. Bringing the system into equilibrium depends both on the amount of energy dispensed to disperse one phase into the other and on the stabilizing elements selected by the skilled person. The size of the droplets forming the dispersed phase of an emulsion is typically in the micrometer range and dictates the white-opaque appearance common to most emulsions. The appearance of the emulsion depends on the size of the dispersed particles, transparency being a characteristic of microemulsions whose size is less than 100 nanometers.

According to the embodiments presented herein, stable and transparent to translucent emulsions can be obtained without any stabilizing agents, with polydispersity relative to the amount of turbulent dissipation energy applied to the mixture.

For example, according to one embodiment, droplet size and zeta potential measurements (known as zetasizer) enable us to characterize stable and transparent essential oil emulsions with average sizes ranging from 450 nanometers to 1.1 microns, without an upstream size reduction system.

According to one embodiment, a method is proposed for preparing a stable emulsion using a magnet cage wherein magnets are arranged and attached in such a way as to force poles of the same sign to remain facing each other. The cage comprises at least one tube or at least one pipe arranged to receive a mixture comprising a hydrophilic element and a hydrophobic element. The at least one tube or the at least one pipe is arranged between the magnets so that the mixture is subjected to the magnetic field.

According to an alternative embodiment or in combination with the above, a method is proposed based on the use of a magnetic field, more particularly the electromagnetic force created in a focal zone by bringing together two magnets of identical polarity and strength. Applying such a force to an aqueous solution containing water-immiscible components both reduces the size of the liquid particles and stabilizes the emulsion that then presents a transparent appearance.

The at least one tube or the at least one pipe can be arranged along a single defined length or can be arranged as a coil between the magnets.

Preferably, the at least one pipe is made of a plastic material, for example, containing polyurethane, for example, “PTFE.”

According to a particular embodiment wherein the mixture is set in motion in front of the magnets, a pump, for example, a diaphragm pump, is provided to vary the exposure parameters of the preparations subjected to the energy source, from static to recirculation.

In one embodiment, four permanent neodymium magnets are provided. For example, the magnets may have the following characteristics:
dimensions 40 mm×30 mm×22 mm, or, for example, dimensions 200×60 for the opposite side of the mixture,
axial magnetization,
inserted in a U-profile 200 mm long and 60 mm wide,
the + (plus) pole is deflected by the sides of the U-shaped profile toward the adhesion surface,
the − (minus) pole is at the surface of the U-profile,
a threaded rod is placed in the center, and
the magnets can be coated with vulcanized elastomer in order to improve adhesion: the indicated force is 4500N, preferably a vulcanized elastomer coating reinforces the anti-slip effect: the sliding force is equivalent to 70% or even 90% of the adhesion force. The total surface area of the magnetic system formed by the permanent magnets is at least 240 cm2, preferentially 0.2 m², and even more preferentially 0.5 m2, and beyond that, for example, 1 m².

Protocol example:
use demineralized water mixed in a beaker with a hydrophobic element according to different proportions: 0.5%, 1%, 3%, 6%, 10%, 20%;
optionally: aspirate and discard a dozen times using an immersed syringe in order to emulsify coarsely: until there is no visible supernatant;
introduce the mixture into the pipe between the magnets and leave to work for 30 minutes to 2 hours.

Example of Lemon grass (Cymbopogon Citratus) essential oil emulsion in water:

TABLE 1

| | | PDI | | | | |
|---|---|---|---|---|---|---|
| | | (poly- | ZETA | | | Dura- |
| | Size | dispersity | (milli- | | | tion |
| | (nm) | index) | volt) | pH | TR | (min) |
| Lemon 3% | 587.7 | 0.489 | −32.4 | 4.95 | Static | 120 |
| Lemon 10% | 857.2 | 0.639 | −29.6 | 7.10 | Recir- | 120 |
| | | | | | culation | |
| Lemon 6% | 867 | 0.714 | −32.9 | 5.47 | Static | 120 |

Table 1 shows the results, including:
the polydispersity index, known as PDI; its value is between 0 and 1: the closer it is to 0, the more monodispersed the emulsion, the closer it is to 1, the more polydispersed it is;
the Zeta potential, which measures the intensity of electrostatic repulsion or attraction between particles in milli volts.

A negative value is predictive or predictive of long-term stability. However, depending on whether the emulsion is anionic or cationic, a high value can also mean future stability.

The 3% and 6% samples show a stable output state with no supernatant.

The 10% sample has a stable output state but shows a slight surface supernatant of microscopic beads that re-mix after gentle agitation. By increasing the duration by around twenty minutes, the output state is also stable without any supernatant.

In addition, the experiments enable us to obtain emulsions with the following advantages:
excellent olfactory preservation of essential oils;
no significant drop in pH (between 5 and 7.5 regardless of the raw materials);
no temperature increase, regardless of the processing times and the oil concentrations;
total transparency;
robustness of the stability of the emulsion with regard to contact with acid, microwaving, for example, a mixture subjected to a power of 750 W or 900 W, in particular, for 30 seconds, using 5 mL of Neroli 3% or Lemon 6%, or freezing, in particular, at minus 18 degrees Celsius, then thawing, or diluting with water, spirit vinegar or ethanol;
with regard to the incorporation rates achieved for essential oils, it is possible to produce natural perfume compositions in water, the compositions being persistent and transparent.

### Example of a Single-Mix Cosmetic Formulation

The conventional technique for producing cosmetic preparations comprises at least an emulsification/stabilization then preservation phase and a post-processing texturing phase to obtain the target galenic formulation notably in terms of long-term stability.

In this experiment, all the components required for the formulation in a single operation were mixed, in this case:

17.96 g water, 1 g CoQ10, 1 g apricot kernel oil, 0.04 g tocopherol, 1 g xanthan, 1 g sodium hyaluronate, 0.08 g potassium sorbate, in a 50 micron PE bag.

For example, the mixture is placed in a bag in static mode between two magnets for 180 minutes to submit to an electromagnetic force as described previously.

After processing, a homogeneous and stable texture is obtained at room temperature. It is transparent. After 5 weeks at 500 in the oven, there is no phase shift, no creaming and a remarkably stable pH:

TABLE 2

| | pH | Stability |
|---|---|---|
| Week 1 | 7.06 | +++ |
| Week 2 | 6.96 | +++ |
| Week 3 | 6.95 | +++ |
| Week 4 | 6.92 | +++ |
| Week 5 | 6.95 | +++ |

Table 2 shows that the method according to the present disclosure enables the production of an emulsion (herein based on water and apricot kernel oil) that remains stable over time.

### According to Various Tests:

If two liquids are immiscible, the formation of a stable emulsion without the addition of any stabilizer has been observed, the exposure time to the magnetic field being proportional to the concentration of the emulsion.

If two liquids are immiscible, a reduction in the size of emulsion particles has been observed without any heating or pre-emulsion device upstream. Surprisingly, the liquid remains cool (19.8 to 20 °C). Even after several hours of exposure and a drop in size to around 500 nm.

If two liquids are immiscible, particularly in the case of perfume concentrates and essential oils in water, a translucent to transparent emulsion is obtained even with average sizes of 500 nm to 1 micron.

### Example: Preserving Properties of the Active Ingredients:

Two reference antioxidants were selected for comparison in PAOT-Score® analysis with a simple formulation with processing: demineralized water and antioxidant, and complex formulation processing with this same antioxidant.

The antioxidants selected are Quercetin at 0.4 g/liter and Catechin at 2 g/liter.

They are used diluted at 5% in demineralized water, i.e., 1 g per 19 g of water.

The first dilution is called the Reference Solution, and is not processed.

The second dilution is processed for 120 min with the method according to the present disclosure, and is referred to as the Processed Reference Solution.

The third dilution is formulated as follows: for 20 g total: 17.96 g demineralized water, 1 g antioxidant, 1 g sweet almond oil, 0.04 g vitamin E, 0.08 g potassium sorbate, 2 g xanthan texturizer. It is called the Processed Formula. It is subjected to the same processing as the Processed Reference Solution.

TABLE 3

| | | Processed | Processed |
|---|---|---|---|
| | Reference | reference | complex |
| Quercetin 0.4 g/liter | solution | solution | formulation |
| PAOT Scores/g of product | 51.89 | 51.79 | 51.87 |

TABLE 4

| | | Processed | Processed |
|---|---|---|---|
| | Reference | reference | complex |
| Catechin 2 g/liter | solution | solution | formulation |
| PAOT Scores/g of product | 52.31 | 52.18 | 52.07 |

It is observed that the processing maintains the activity of the native antioxidant molecule at the maximum, without alteration or overactivation. This experiment validates the fact that the energy applied to the solutions does not alter the nature of the active compounds.

### Example: Qualifying the Energy of the Applied Magnetic Field

In a room at 20.7 °C, an 11 g bag of water was placed at 60 °C. The bag is placed between two magnets in contact with these magnets. After 5 minutes of processing (exposure to the magnetic field), the water temperature is 410 (control).

Next, four tests are carried out by placing a bag of 11 g of water at 60 °C in the pilot, varying the distance between the magnets.

TABLE 5

| | | |
|---|---|---|
| Test no. | Distance | T°/5 mn |

| | | | |
|---|---|---|---|
| 1 | 0.5 | cm | 21°8 |
| 2 | 1 | cm | 27°9 |
| 3 | 1.5 | cm | 32°1 |
| 4 | 2 | cm | 37°2 |

Surprisingly, a simple correlation emerges: 5° for every 0.5 cm difference are “lost.”

The best case is when the field is in contact, preferably equidistant, with the system to be processed.

Moreover, processing time and quality are better when the liquid is in a small volume.

These observations support the hypothesis whereby the magnetic field generates turbulent vortex-like energy, and, in particular, quantum vortex energy, capable of cooling a liquid.

### Example: Demonstrating Turbulent Kinetic Energy Dissipation Using the Method According to the Present Disclosure.

### First Test:

Room temperature 210
11.25 g of demineralized water heated to 68° is placed in a 50 micron PE bag for food use and placed in the presence of the magnetic field in accordance with the present disclosure. The bagging temperature is T0.

TABLE 6

| | |
|---|---|
| T0 | 68° |
| T0 + 5 mn | 25.2° |
| T0 + 10 mn | 22.3° |
| T0 + 15 mn | 21.6° |
| T0 + 20 mn | 21.2° |
| T0 + 25 mn | 21.2° |

Repeating with Controls:

Under the same experimental conditions for the bag to be processed, two controls to measure the differences in temperature decrease kinetics were added: controls left at room temperature are one bag of 11.25 g and a glass beaker of 11.25 g.

TABLE 7

| TEST 1 | Processed bag | Bag at room temp. | Beaker at room temp. |
|---|---|---|---|
| T0 | 47° | 47° | 47° |
| T0 + 10 | 21.6° | 33° | 33.3° |
| T0 + 20 | 21.1° | 27.5° | 28.5° |
| T0 + 30 | 20.9° | 24.8° | 26° |
| T0 + 40 | 20.9° | 23.4° | 24.7° |
| T0 + 50 | 20.8° | 22.7° | 23.2° |

These results confirm the cooling effect obtained by processing with the method according to the present disclosure.

### Case of Inverse Emulsion:

The first phase of the experiment consists of identifying the feasibility of preparing a water-in-oil inverse emulsion with readable experimental parameters.

The chosen fat is cocoa butter as it has a melting point of 37 °C and is therefore easy to handle.

A mixture of cocoa butter and water is prepared at 55 °C.

The sample is weighed before processing, i.e.:
one zipped PE bag: 0.36 g
demineralized water: 4.20 g
cocoa butter: 4.11 g, for a total of 8.47 g

The ratio of raw material to processed material is 50.5% water to 49.5% cocoa butter.

After 30 minutes of processing, the preparation is solidified (i.e., back above the melting point).

It is important to compare the cooling process, which is much slower than in the case of an aqueous preparation. This confirms that viscosity is a major criterion in the dispersion of turbulent energy (lower Reynolds number).

The sample is also weighed after treatment:
one zipped PE bag: 0.36 g
water: 3.36 g
solidified cocoa butter: 4.75 g, for a total of 8.47 g

The ratio of raw material to processed material is 43.5% water to 58.5% textured cocoa butter.

The structure of the solid obtained versus the control is less brittle and loaded with water.

Of course, the present disclosure is not limited to the described embodiments, and it is possible to add variants thereto without departing from the scope of the invention as defined by the claims.

1. A method for preparing a stable emulsion from a mixture comprising at least one hydrophilic fraction and at least one hydrophobic fraction, at least one of the two fractions being liquid, the other being liquid or solid, by generating a turbulent flow applied to the mixture, the turbulent flow being generated and maintained by at least one magnetic force created by at least two identical magnetic poles.

2. The method of claim 1, wherein the turbulent flow is generated and maintained solely by at least one magnetic force.

3. The method of claim 2, wherein the turbulent flow is obtained by dissipating at least one magnetic force or at least one magnetohydrodynamic wave in the mixture.

4. The method of claim 3, wherein the turbulent flow is achieved by dissipating the induction forces applied as kinetic energy, with a Reynolds number greater than 100,000.

5. The method of claim 4, wherein the turbulent flow is generated and maintained by at least one magnetic force created by at least two magnetic poles positioned facing each other.

6. The method of claim 5, wherein the mixture is placed equidistant from the magnetic poles.

7. The method of claim 6, wherein the turbulent flow is generated and maintained by at least one magnetic force generating at least 100 millitesla (or 1000 gauss).

8. The method of claim 7, wherein no stabilizing agent is added.

9. The method of claim 8, wherein the hydrophilic fraction is an aqueous solution and the hydrophobic fraction comprises at least one vegetable oil or at least one essential oil.

10. The method claim 1, wherein the stable emulsion comprises a cosmetic or aromatic composition.

11. A stable emulsion formed by a method according to claim 1, wherein the stable emulsion includes particles greater than 400 nanometers in size.

12. The method of claim 1, wherein the turbulent flow is obtained by dissipating at least one magnetic force or at least one magnetohydrodynamic wave in the mixture.

13. The method of claim 1, wherein the turbulent flow is achieved by dissipating the induction forces applied as kinetic energy, with a Reynolds number greater than 100,000.

14. The method of claim 1, wherein the turbulent flow is generated and maintained by at least one magnetic force created by at least two magnetic poles positioned facing each other.

15. The method of claim 1, wherein the mixture is placed equidistant from the magnetic poles.

16. The method of claim 1, wherein the turbulent flow is generated and maintained by at least one magnetic force generating at least 100 millitesla (or 1000 gauss).

17. The method of claim 1, wherein no stabilizing agent is added.

18. The method of claim 1, wherein the hydrophilic fraction is an aqueous solution and the hydrophobic fraction comprises at least one vegetable oil or at least one essential oil.
